(19) United States
(12) Patent Application Publication
(10) Pub. No.: US 2017/0239444 A1
(43) Pub. Date: Aug. 24, 2017

(54) CATHETER GRIP

(71) Applicant: Steven Parker, Spokane, WA (US)

(72) Inventor: Steven Parker, Spokane, WA (US)

(21) Appl. No.: 15/051,513

(22) Filed: Feb. 23, 2016

(51) Int. Cl. A61M 25/01; A61B 5/00

(52) U.S. Cl. CPC A61M 25/013; A61B 5/6852; A61N 1/056

(57) ABSTRACT

The present disclosure provides a catheter grip device that secures to a catheter shaft and provides easier manipulation of the catheter shaft for the user. For example, the catheter shaft, such as for an Electrophysiology catheter, may insert into a patient's blood vessel. From here, the user, such as the physician, may need to manipulate, re-position, rotate, or otherwise maneuver a distal end of the catheter shaft while inserted inside the patient's blood vessel. In this regard, by the catheter grip being securely attached to the catheter shaft, the user is able to manipulate, rotate, etc. the catheter shaft by rotating and manipulating the catheter grip. The catheter grip reduces strain on the various muscles of the user's hand, and also provides more accurate manipulation of the catheter shaft.

### BACKGROUND

Patients undergo Electrophysiology tests in order to identify the cause of an arrhythmia. During an electrophysiology test, a catheter shaft is inserted into the patients blood vessel, such as via the groin, in which a doctor sends electrical pulses through the catheter shaft that cause the heart to beat at various speeds. The heartbeat rhythms are picked-up and recorded by a catheter device (e.g., cardiac mapping), and the doctor is able to identify the cause of the arrhythmia, which may then be destroyed via a catheter ablation.

The catheter shaft is inserted and controlled by a proximal handle attached to the catheter shaft. An electrical cable is inserted into the proximal end of the handle to control the electrical pulses sent through the catheter. In order to manipulate a distal end of the catheter shaft, the doctor may manipulate the catheter handle and/or grasp the catheter shaft itself and rotate it, such as by using an index finger and thumb. By rotating as such, strain and muscle soreness or tightness may result because the doctor has to continue rotating the catheter shaft using only the two fingers throughout the procedure. Strain on doctor's muscle may affect the doctor's ability to adequately control the catheter shaft.

### SUMMARY

A catheter grip device that provides easier control and thereby less strain on the doctor's muscles is disclosed herein. The catheter grip is secured to the electrophysiology catheter shaft with pressure from a receiving portion attached to a threaded cap, wherein the threaded cap rotates about the threads of the receiving portion, thereby securing the catheter shaft in place. Once the catheter shaft is secured to the catheter grip, the doctor is able to grasp the catheter grip in order to rotate the catheter shaft, as opposed to only being able to grip the catheter shaft itself. In this regard, the doctor can rotate the catheter grip when he or she needs to manipulate the distal end of the catheter shaft. The doctor can move the distal end of the catheter shaft forward and backward relative to the patient and/or rotate the distal end of the catheter shaft within the blood vessel of the patient. The ability to manipulate the catheter shaft using the catheter grip may also allow the muscles in the wrist, forearm, and elbow to absorb the strain that would typically be solely on the muscles within the hand of the doctor, such as the opponens pollicis and adductor pollicis.

A catheter grip is disclosed herein, the catheter grip including a cap having an interior cavity; a grip; and a receiving portion extending from the grip, the receiving portion having an insertion area configured to receive a catheter shaft, wherein the interior cavity of the cap is fastened to the receiving portion of the grip.

As a further example, the grip has a first side and a second side, the receiving portion extending from the first side and a gripping portion being on the second side of the grip. As another example, the insertion area of the receiving portion is a recess, the recess extending perpendicular to the receiving portion. As another example, at least a portion of the recess includes a rubber insert to further secure the catheter grip in place. In that example, the recess extends from a first end of the receiving portion to a second end of the receiving portion. As another example, when a catheter shaft is received within the recess of the receiving portion such that the catheter shaft rests on a recess base of the recess, a circumferential end of the cap contacts the catheter shaft such that the catheter shaft is positioned between the end of the cap and the recess base, thereby securing the catheter shaft in place. As a further example, the insertion area is a bore that extends longitudinally from a first end of the receiving portion to a second end of the receiving portion. In that example, the bore further extends through the grip. In a further example, the cap includes a cap bore that corresponds to the bore of the receiving portion when the receiving portion and the cap are at least partially fastened to each other. As another example, the cap includes a cut-out that defines an opening on at least a portion of the cap threads of the cap, the opening corresponding to at least a width of a catheter shaft, and the cut-out extending to the cap bore of the cap. As another example, the receiving portion of the grip includes flanges surrounding the bore, the flanges being configured to adjust a size of the bore, wherein the flanges have a resting position and a tightening position, wherein in the resting position the size of the bore has a first diameter adapted to receive the catheter shaft, and in the tightening position the size of the bore has a second diameter adapted to come into contact with the catheter shaft and thereby secure the catheter shaft in place. As an additional example, the cap is fastened to the receiving portion via at least one of threads, tab and notch, and teeth that secure to a pawl. As a further embodiment, a top of the receiving portion is tapered.

As another embodiment, an apparatus is disclosed herein, the apparatus including a grip; a first flanges extending from a first side of the grip; and a second flanges extending from a second side of the grip, the second side being opposite the first side; and a bore extending from the first flanges to the second flanges, wherein the bore is configured to receive a catheter shaft, and the first and second flanges are adapted to shift between a resting position and a tightening position.

As a further example of the apparatus, the apparatus includes a first cap having a first cap bore; a second cap having a second cap bore; and when the first cap and the second cap are mated with the first flanges and the second flanges, respectively, and the position of the first cap bore and the second cap bore correspond to the bore of the first flanges and the second flanges.

As another embodiment, a kit is disclosed, the kit including a first cap, the first cap having a first interior cavity; a grip; and a receiving portion extending from the grip, the receiving portion having an insertion area configured to receive a first catheter shaft, wherein the interior cavity of the cap is fastened to the receiving portion of the grip.

The kit further includes a second cap, wherein the second cap has a second interior cavity, the second interior cavity being a different size from the first interior cavity. As a further example, the kit includes the first catheter shaft. In another example, the first cap has a cap bore, and when the cap is fastened to the receiving portion, a position of the cap bore corresponds to an insertion area of the receiving portion.

### BRIEF DESCRIPTION OF THE DRAWINGS

FIG. 1 is a perspective view of a grip and a cap of a catheter grip in accordance with aspects of the present disclosure.

FIG. 2 illustrates a receiving recess of a receiving portion of the grip in accordance with aspects of the present disclosure.

FIG. 3 depicts a catheter shaft prior to insertion in the receiving recess in accordance with aspects of the disclosure.

FIG. 4 is a perspective view of an internal portion of the cap of the catheter grip in accordance with aspects of the present disclosure.

FIG. 5 is a perspective view of the grip mating with the cap in accordance with aspects of the present disclosure.

FIG. 6 illustrates the catheter grip with a catheter and a catheter shaft in accordance with aspects of the disclosure.

FIGS. 7A-J depict alternative embodiments of the grip in accordance with aspects of the present disclosure.

FIGS. 8A-E are perspective views of a single-sided flange catheter grip with a slit in accordance with aspects of the present disclosure.

FIGS. 9A-C are perspective views of the single-sided flange catheter grip without the slit in accordance with aspects of the present disclosure.

FIGS. 10-C are perspective views of a double-sided flange catheter grip in accordance with aspects of the present disclosure.

FIGS. 11A-B illustrate alternative embodiments to fasten the cap to the receiving portion in accordance with aspects of the present disclosure.

### DETAILED DESCRIPTION

The aspects, features and advantages of the present disclosure will be appreciated when considered with reference to the following description of preferred embodiments and accompanying figures. The following description does not limit the disclosure; rather, the scope is defined by the appended claims and equivalents. While certain processes in accordance with example embodiments are shown in the figures as occurring in a linear fashion, this is not a requirement unless expressly stated herein. Different processes may be performed in a different order or concurrently.

The present disclosure describes a catheter grip device that securely attaches to a portion of the catheter shaft as an add-on component, and provides easier gripping and manipulation of the catheter shaft by an individual, such as a doctor. The catheter grip includes a grip and a cap. A receiving portion extends from the grip and cap threads are positioned on an interior cavity of the cap. A portion of the catheter shaft may be placed into a receiving recess of the receiving portion of the grip, and the cap threads of the cap may rotate about the receiving portion of the grip. The cap may continue to rotate about the receiving portion and the cap threads may rotate until the catheter shaft is securely positioned between a bottom end of the receiving recess and a circumferential end of the cap. In this regard, the user may rotate the cap about the receiving portion until rotating becomes difficult, e.g., the user determines that the catheter shaft is securely in place within the catheter grip. Additionally, different caps may be implemented and used that have different internal depths so that the cap cannot be rotated beyond the desired depth depending and based on the catheter shaft thickness, this way the user cannot damage the catheter. At this point, when the user needs to manipulate movement of the catheter shaft, grabbing the catheter grip provides the doctor with a more robust grip to do so. In this regard, the catheter grip provides easier gripping of the catheter shaft and also easier manipulation and movement of the catheter shaft.

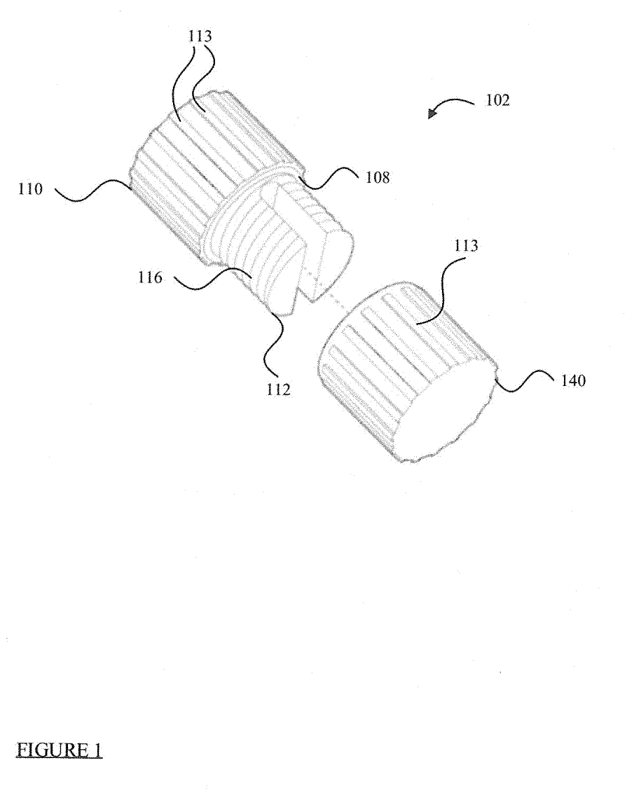

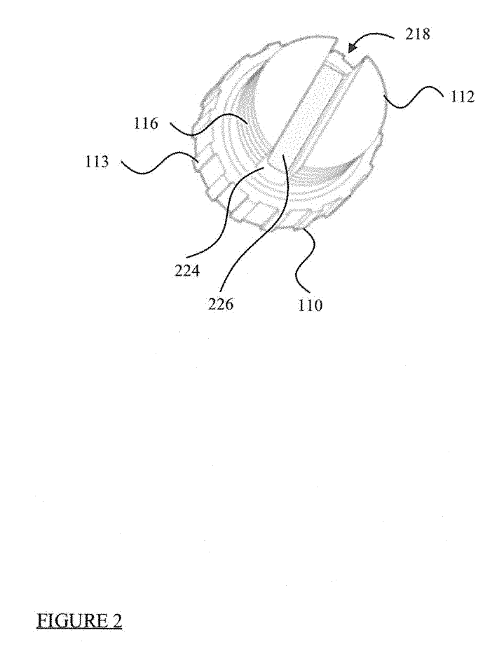

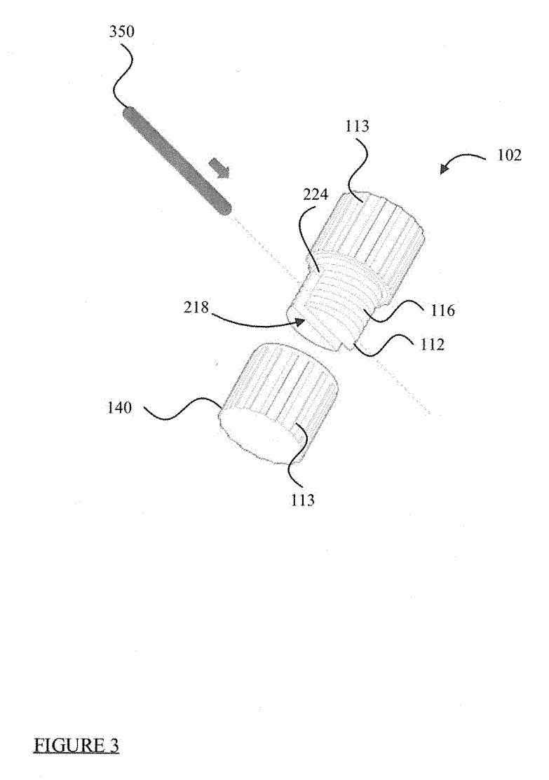

FIG. 1 illustrates catheter grip 102 according to the above disclosure. Catheter grip 102 includes grip 110 that mates with cap 140. For instance, grip 110 includes receiving portion 112 that extends outward from a base 108 of grip 110. As illustrated in FIG. 1, receiving portion 112 includes threads 116 on an outer surface thereof in order to mate with cap 140. Furthermore, grip 110 and cap 140 may include ridges 113 thereon to provide additional support when a user grasps grip 110 and cap 140. It should be understood that although the figures herein depict catheter grip 102 utilizing threads to mate receiving portion 112 with cap 140, other fastening mechanisms are also possible as discussed in further detail below, such as a tab and notch mechanism, friction fit, press-fit, etc.

Grip 110 and cap 140 may be comprised of plastic or metal. Even further, grip 110 and cap 140 may be comprised of any number of materials that provide sufficient structure to form the requisite pieces, such as polycarbonate, polypropylene, Acrylonitrile-Butadiene-Styrene, thermoplastic elastomer, polyethylene, cyclo olefin polymer, silicone, polyetherimide, polyetheretherketone, polysulfone, stainless steel, or polytetrafluoroethylene.

With respect to the catheter shaft, the catheter shaft may be made from woven Dacron braiding and or an extrusion process using synthetic materials like polyethylene. The internal components consist of nitinol wires and electrical wires. For example, the nitinol wires may be connected to a steering mechanism and to a distal end of the catheter shaft and be used to deflect the catheter in variable a radius in plane with the catheter shaft. Additionally, the electrical wires may be connected to a proximal plug and distal individual electrodes and configured to pace, sense, and deliver RF energy to tissues of the body.

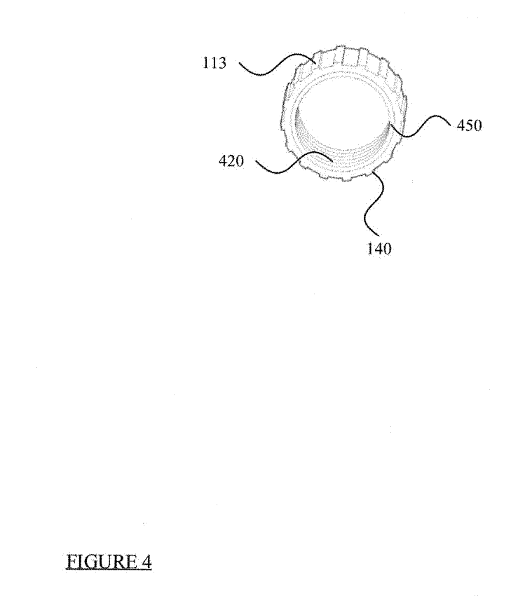

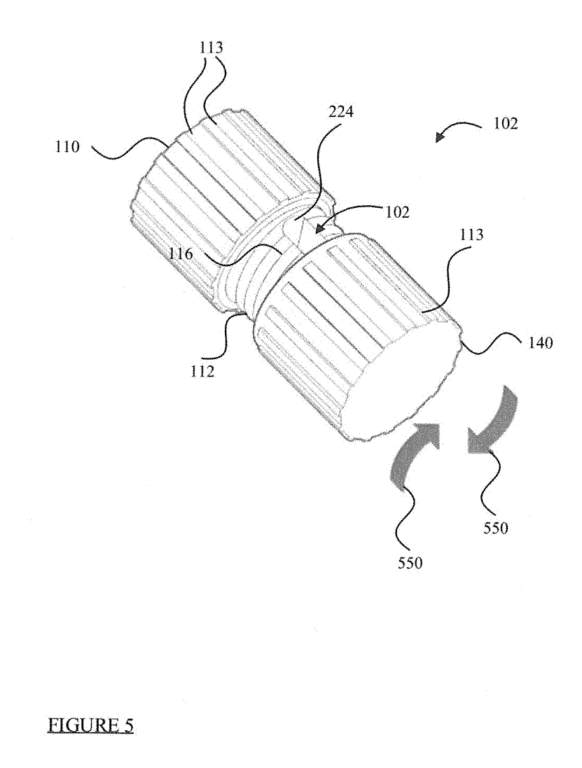

As depicted in FIG. 2, receiving portion 112 includes an insertion area in the form of receiving recess 218. Receiving recess 218 is defined by the walls of receiving portion 112, which surround receiving recess 218. A bottom of receiving recess 218 may extend to recess base 224. As another example, recess 218 may extend any distance between recess base 224 and top surface 220 of receiving portion 112.

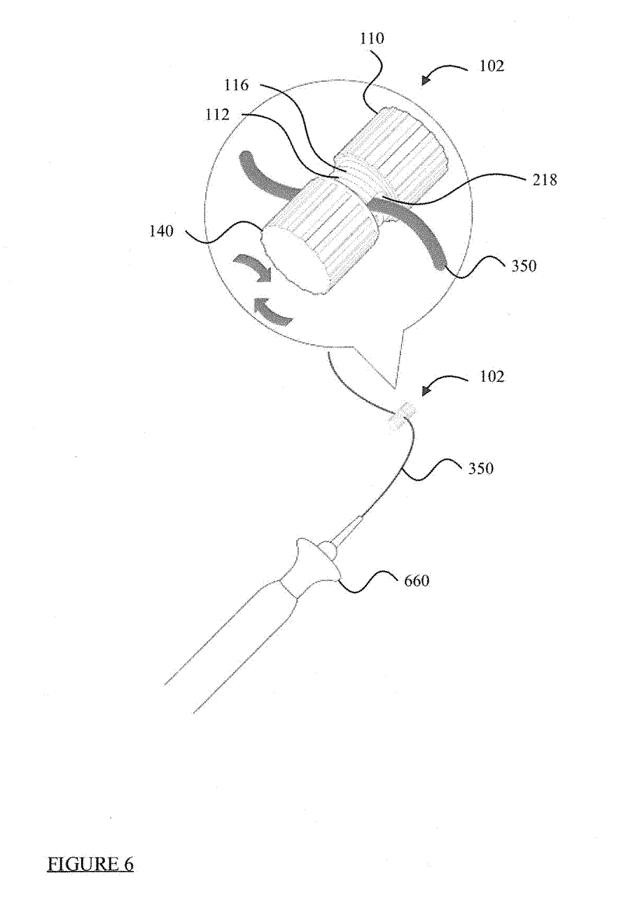

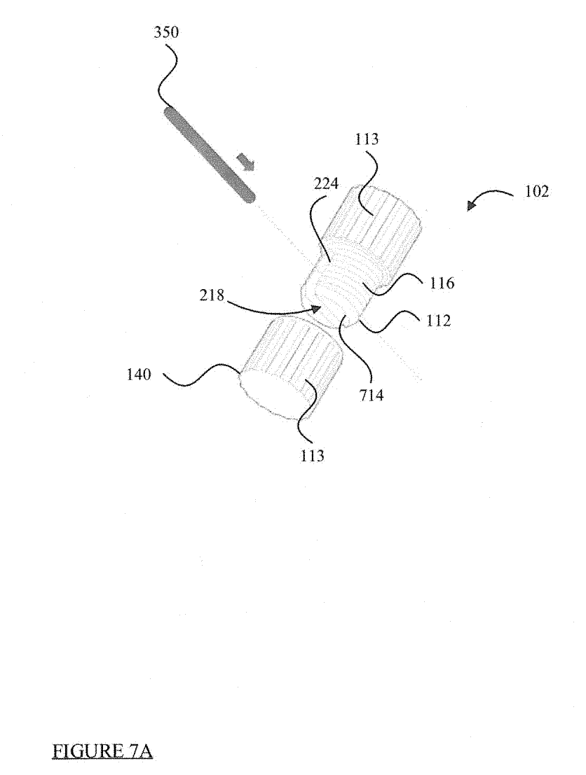

Catheter shaft 350 may be placed within receiving recess 218, such that catheter shaft 350 extends perpendicular to grip 110. As one example, catheter grip 102 may be designed for Electrophysiology Catheters having a diameter of One to Twenty-Two on the French Gauge. For instance, as shown in FIG. 3 catheter shaft 350 can be inserted in one of the sides of receiving recess 218. Catheter shaft 350 may be connected to a catheter (not shown) on a proximal end of catheter shaft 350. The housing of receiving portion 112 may define and dimension receiving recess 218 such that catheter shaft 350 fits within receiving recess 218. For example, an external body of catheter shaft 350 may correspond to internal walls or surfaces of receiving recess 218, as defined by the housing of receiving portion 112. It should be understood that receiving recess 218 and catheter shaft 350 are not restricted to any particular size, shape, etc. Rather, receiving recess 218 of catheter grip 102 may be designed to contemplate any sized catheter shaft.

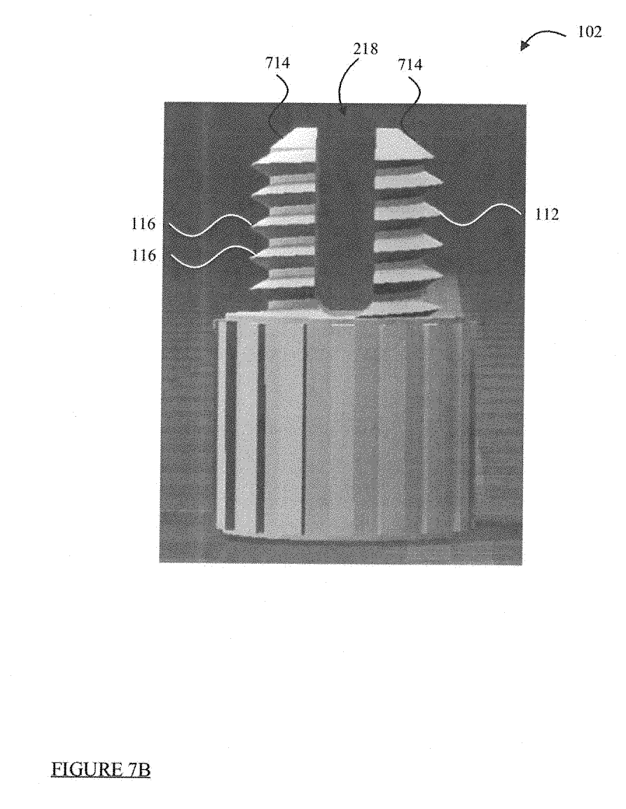

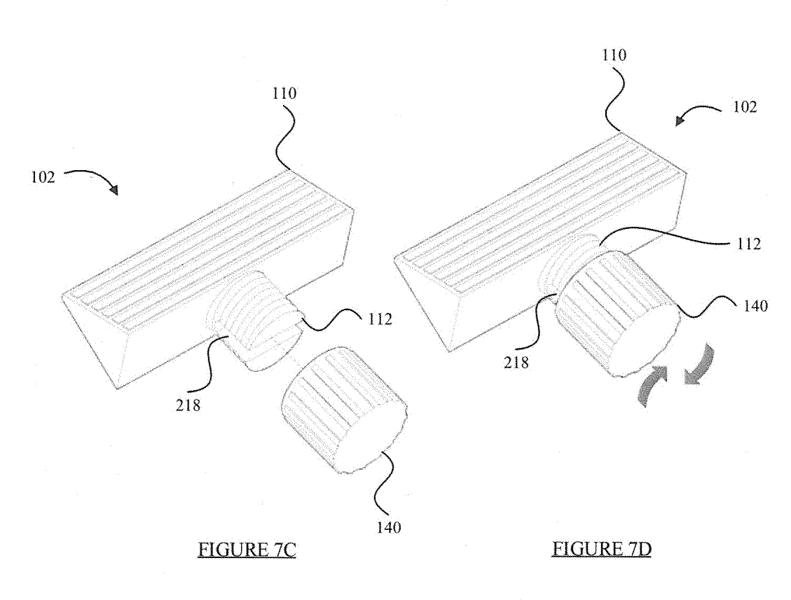

After catheter shaft is placed within receiving recess 218 of receiving portion 112, cap 140 may be fastened to receiving portion 112 via threads 116. In this regard and as shown in FIG. 4, cap 140 includes cap threads 420 that rotate about threads 116 in order to fasten the two pieces together. Cap threads 420 are positioned on an inside portion of cap 140, such that the inside portion of the cap rotates about receiving portion 112. In this regard, receiving portion 112 is inserted inside an interior cavity of cap 140 once the two pieces are fastened together. As another example, cap 140 may be partially fastened to receiving portion 112 before catheter shaft 350 is inserted therein. For example, cap 140 may be fastened halfway through receiving portion 112 and catheter shaft 350 can still be inserted within receiving recess 218. As a further example, cap 140 and receiving portion 112 may be fastened to each other to any degree before catheter shaft 350 is inserted, as long as catheter shaft 350 is still insertable within receiving recess 218. In the situation where cap 140 and receiving portion 112 are fastened to each other to the point where catheter shaft is blocked from being inserted into receiving recess 218, a user may unfasten cap 140 from receiving portion 112 to provide the user with room to insert catheter shaft 350.

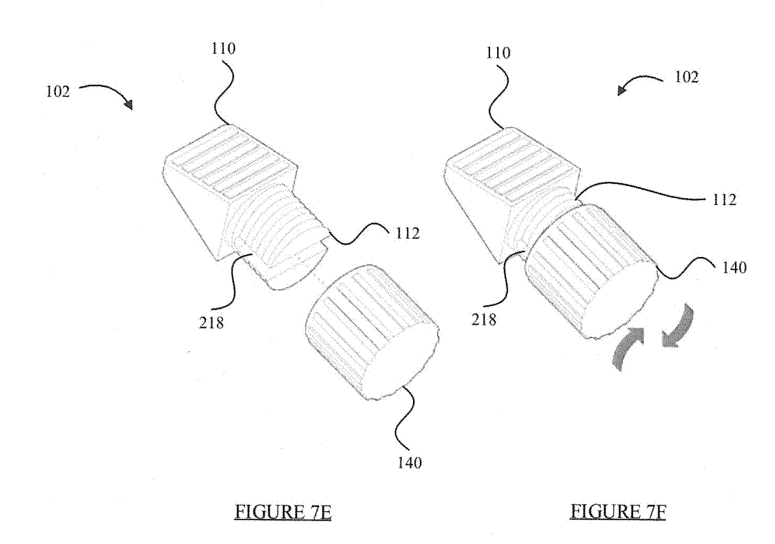

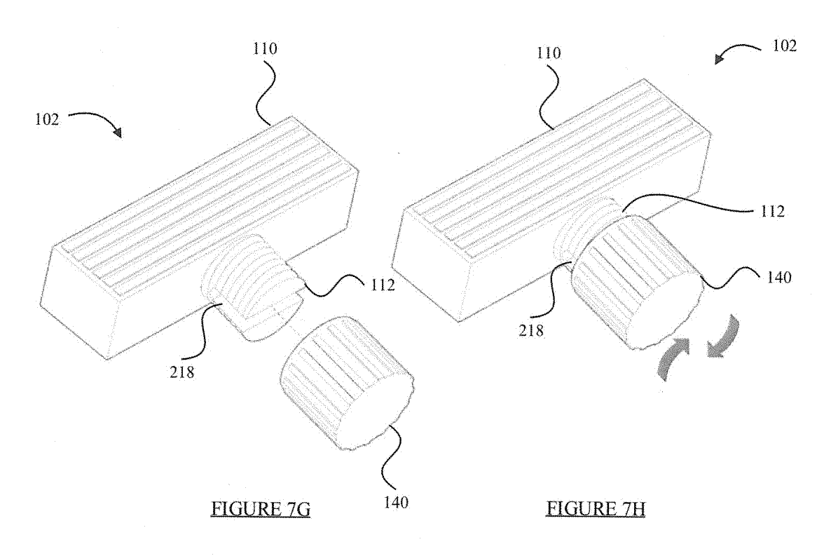

Cap threads 420 of cap 140 may rotate about threads 116 of receiving portion 112 until catheter shaft 350 is securely positioned between recess base 224 of receiving portion 112 and a circumferential end 450 of cap 140. In this regard, cap 140 should be fastened to receiving portion 112 in a manner that sturdily secures catheter shaft 350 in place and prevents catheter shaft 350 from freely moving. FIG. 5 illustrates cap 140 mated with receiving portion 112 of grip 110. As shown by arrows 550 cap 140 rotates clockwise in order to fasten cap threads 420 to threads 116 of receiving portion 112. Cap 140 may be rotated continuously until circumferential end 450 of cap 140 engages catheter shaft 350. For example, circumferential end 450 may continue to push catheter shaft 350 toward recess base 224. In that regard, cap 140 may continue to be rotated about receiving portion 112 until catheter shaft is securely positioned against a recess base 224 of recess 218 and circumferential end 450 of cap 140. In this regard, different sized and shaped catheter shafts can be used with the single device by the user being able to adjust the degree of fastening cap 140 to grip 110.

FIG. 6 depicts a perspective view of catheter grip 102 fully assembled with catheter 660 and catheter shaft 350. As illustrated in FIG. 6, catheter shaft 350 is inserted perpendicular to catheter grip 102. In addition, catheter grip 102 covers only a small portion of catheter shaft 350, thus not being obstructive or a hindrance to the user. Catheter grip 102 may be positioned at any point on catheter shaft 350, so long as there is sufficient length of catheter shaft 350 to be inserted within a blood vessel of a patient. When catheter grip 102 is fully assembled as in FIG. 6, the user is able to grip catheter grip 102 with their hand and fingers, thereby providing them ease to manipulate, rotate, insert, remove, etc. catheter shaft 350 when inserted within the patient.

Without catheter grip 102 the user may be restricted to grasping catheter shaft 350 itself when manipulating or repositioning catheter shaft 350 within the patient. For example, typically the user may use his or her thumb and index finger to apply pressure to catheter shaft 350 in order to manipulate a distal end of catheter shaft 350. By only gripping catheter shaft 350 this may cause additional strain to the user's hand muscles, such as the Adductor Pollicis and Opponens Pollicis muscles. Catheter grip 102 helps remove strain from the various muscles in the hand of the user by allowing the user to grasp a more robust device, catheter grip 102, which is not only easy to manipulate but may also allow the user to be more accurate and careful when manipulating catheter shaft 350 within the patient. In this regard, catheter grip 102 is not only useful in terms of reducing strain on the muscles of the user, but also increases safety for the patient.

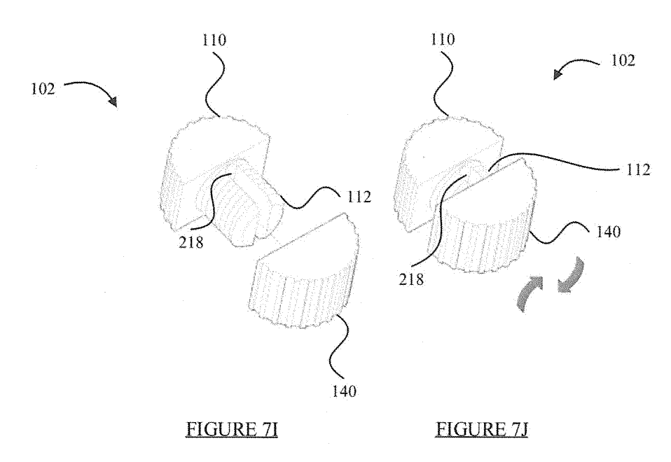

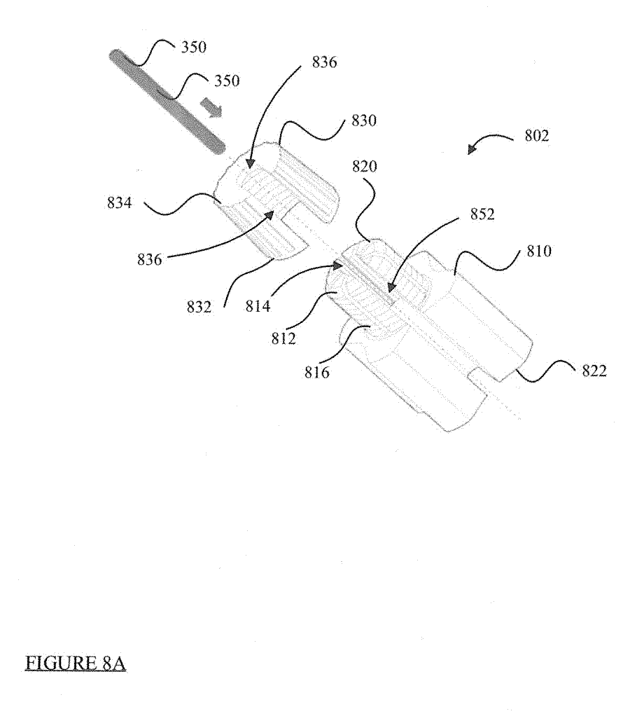

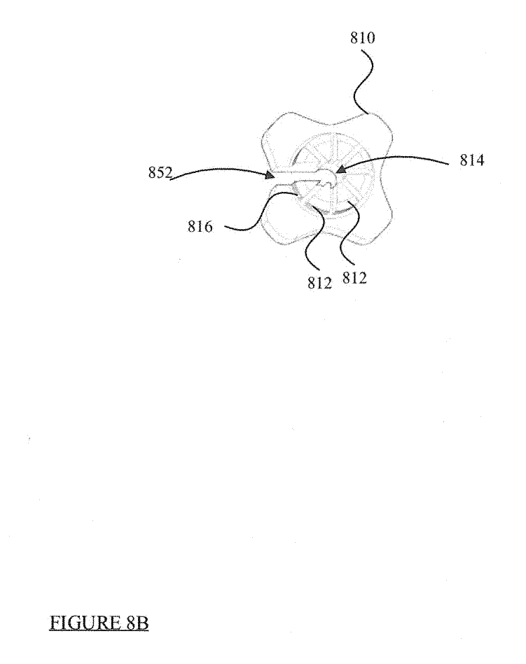

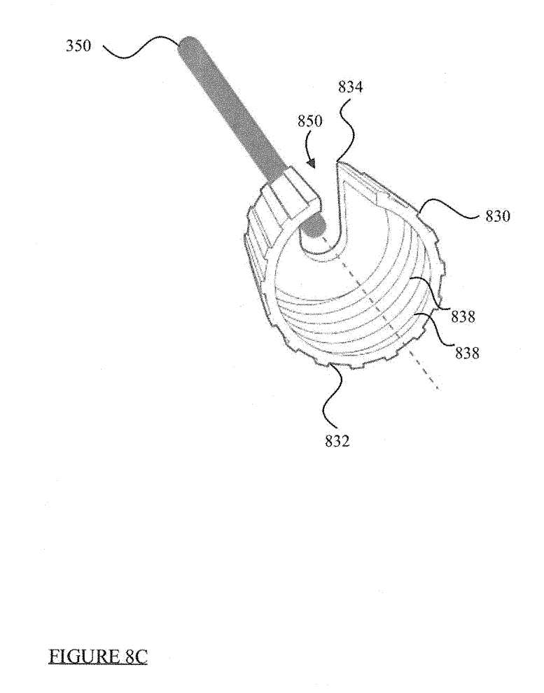

FIGS. 7C-J depict alternative designs and embodiments of catheter clamp 102 as discussed above. For example, with respect to FIGS. 7A-B receiving portion 112 further includes taper 714 at a top portion thereof. In this regard, taper 714 aids in the placement of receiving portion 112 being inserted and mated with cap 140.

As additional examples, the catheter clamps depicted in FIGS. 7C-J illustrate different designs of grip 110, the grips of which being different shapes and sizes, such as some being more robust than others. In this regard, the user may use one of the alternative catheter clamps of FIGS. 7C-J to have even more grip of the catheter clamp, and thereby better control over the catheter shaft.

FIGS. 7C-D illustrate one example of the grip of the catheter grip forming a three-dimensional triangular shape that extends parallel to the receiving recess and thereby parallel to the catheter shaft, when inserted. In this regard, the design in FIG. 7C is more robust than the grip 110 depicted in FIG. 1, thereby providing even more material for the user to grasp. FIGS. 7E-F illustrate another example of the catheter grip, that often forms a three-dimensional triangular shape as in FIGS. 7C-D, albeit includes less substance. FIGS. 7E-F may be similar in size to grip 110 in FIG. 1, but forms a different shape. As a further example, FIGS. 7G-H depict grip in a three-dimensional rectangular fashion. As shown in FIGS. 7G-H, the grip forms a three-dimensional rectangle beyond the receiving portion, such that the grip extends parallel to the receiving recess and thereby parallel to the catheter shaft, when inserted. In another example, FIGS. 7I-J illustrate grip 110 and cap 140 forming a semi-circular design. In this regard, when grip 110 and cap 140 are mated, as shown in FIG. 7J, the pieces form almost a unified circle. The design in FIGS. 7I-J may maximize the ability of the user to rotate the pieces about each other. As depicted in FIGS. 7A-J, the grip may include ridges as well to provide additional support to the user.

FIGS. 7A-J are exemplary of different designs of catheter grip 102, and should not be construed as limiting the different designs of catheter grip 102. For example, although FIGS. 7C-D and 7G-H illustrate grip extending parallel to the receiving recess, and thereby the catheter shaft when inserted, the grip may extend in any direction 360° around the receiving portion or receiving recess. For example, the grip may extend diagonal to the receiving recess, perpendicular to the receiving recess, off-centered from a parallel axis of the receiving recess, etc. As a further example, grip may form various shapes and sizes in addition to extending in various directions, such as forming a shape that resembles an X. Other variations and shapes are also possible.

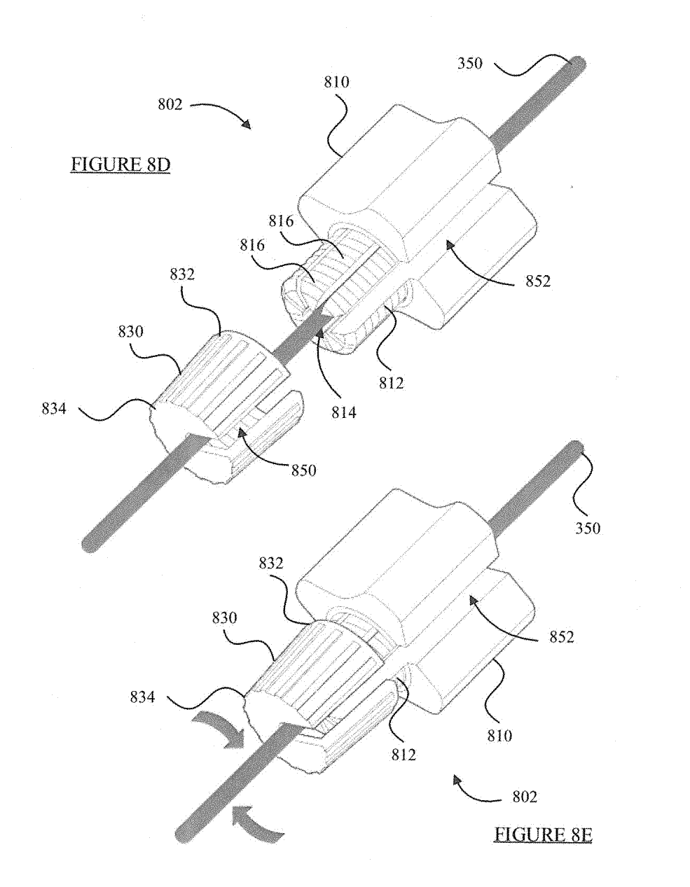

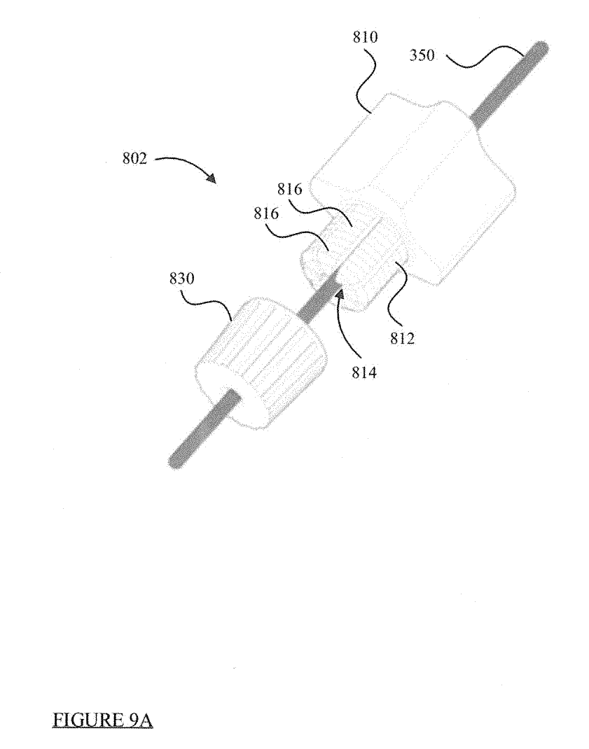

As an alternative embodiment, FIGS. 8A-E depict single-sided flange catheter clamp 802, which includes flanges 812 that surround grip bore 814 of flange grip 810. In this regard, the insertion area is formed by grip bore 814 instead of the receiving recess as discussed above. As illustrated in FIG. 8A, catheter shaft 350 inserts within cap bore 836 and then into grip bore 814 of flange grip 810, grip bore 814 extending from a first end 820 to a second end 822 of flange grip 810, thereby allowing catheter grip 350 to extend completely through flange grip 810.

Single-sided flange catheter grip 802 includes flanges 812 that clamp down on catheter shaft 350, thereby securing catheter shaft 350 in place. FIG. 8B depicts flange grip 810, which includes multiple flanges 812, grip bore 814, threads 816 and grip slit 852. As shown in FIG. 8B, flanges 812 include threads 816 thereon that corresponding cap threads 838 of cap 830 fasten with. In addition, grip slit 852 has a width that corresponds with or is slightly wider than catheter shaft 850, so that catheter shaft 350 can be inserted therein. Grip slit 852 may provide easier insertion of catheter shaft 350, as discussed in greater detail below.

FIG. 8C depicts flange cap 830, which includes cap threads 838 that correspond with threads 816 on flange grip 810. When corresponding cap threads 838 of cap 830 rotates about threads 816 on flanges 812, flanges 812 tighten and reduce the size of grip bore 814. In this regard, flanges 812 have a resting position and a tightening position. In the resting position grip bore 814 has a first diameter, and in the tightening position grip bore 814 has a second diameter that is smaller than the first diameter. In the tightening position, the radius of the second diameter may correspond to an external body of catheter shaft 350 in order to secure catheter shaft 350 in place via flanges 812. FIGS. 8D-E illustrate catheter shaft 350 inserted and assembled with single-sided catheter grip 802.

For example, the more that cap 830 is rotated about flanges 812, the more secure catheter shaft 350 will be within grip bore 814. Lower portion 832 of cap 830 may be wider than an upper portion 834 of cap 830. In this regard, lower portion 832 may be considered wider portion 832 and upper portion 834 may be considered narrow portion 834 of cap 830. Therefore, the more cap 830 is rotated about threads 816 of flanges 812, narrow portion 834 of cap 830 will squeeze or contract flanges 812, thereby securing flanges 812 closer and securely to catheter shaft 350.

As depicted in FIGS. 8A-E, cap 830 may also include cap slit 850 that provides the user with another method to insert catheter shaft 350 therein. Cap slit 350 and grip slit 352 define a size and shape that correspond to or are larger than the size and shape of the external housing of catheter shaft 350. Cap slit 350 and grip slit 352 allow the user to insert catheter shaft 350 into single-sided catheter grip 802 while cap 830 is attached to flanges 812, so long as flanges 812 are not tightened to a degree that obstructs grip slit 852. Alternatively, if cap 830 is not attached the user may insert catheter shaft 350 into flange grip 810 perpendicularly via grip slit 852 as an alternative or an additional option to using grip bore 814.

As discussed above, flanges 812 tighten around catheter shaft 350 by narrower portion 834 of cap 830 contracting the flanges to reduce the size of flange bore 814 until flanges 812 come into contact with catheter shaft 350. Thus, if cap 830 is only slightly rotated about threads 816 of flanges 812, flanges 812 may not be contracted enough to secure catheter shaft 350 in place, reduce the size of the diameter of grip bore 814, or block off grip slit 852. Therefore, catheter shaft 350 can still be inserted, removed, or otherwise adjusted in grip bore 814 or grip slit 852, because the diameter of grip bore 814 is still wide enough to receive catheter shaft 350 and flanges 812 are not contracted enough to obstruct grip slit 852. In that scenario, the user may leave cap 830 on flanges 812, at least slightly, thereby allowing the user to insert catheter shaft 350 into any one of cap bore 836, cap slit 850, grip bore 814 and grip slit 852. From here, the user may tighten cap 830 about threads 816 of flanges 812 until flanges 812 are securely tightened about catheter shaft 350.

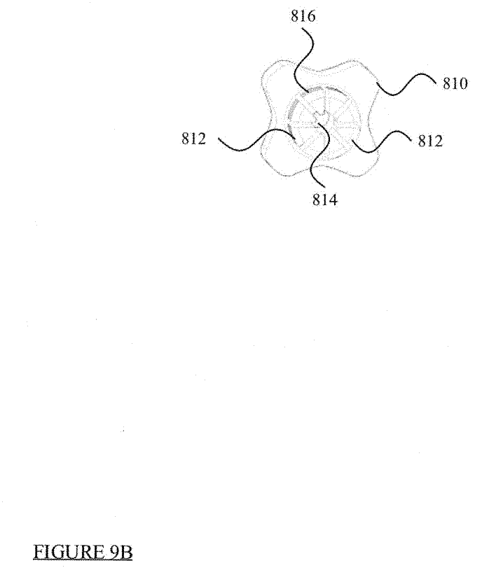

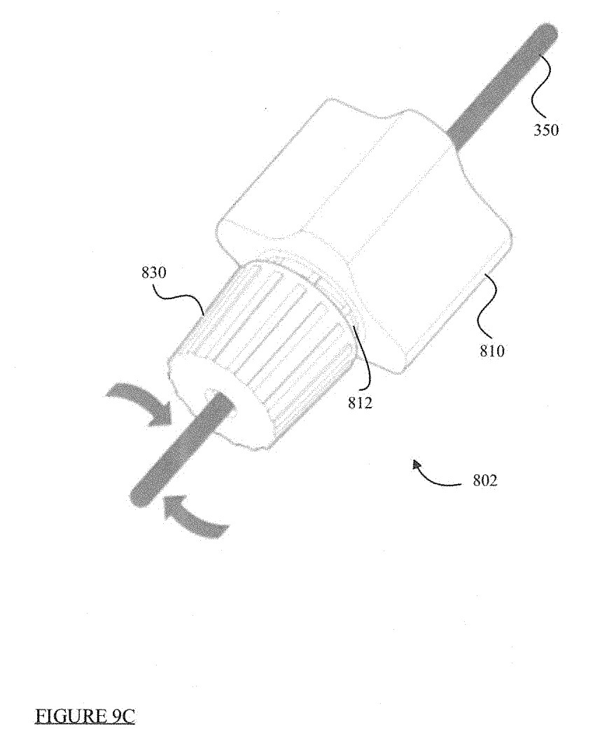

FIGS. 9A-C illustrate single-sided flange catheter grip 802 described above, only without cap slit 850 and grip slit 852. In this regard, catheter shaft 350 can only be inserted via cap bore 836 and grip bore 814, as discussed above.

Although the figures illustrate there being a certain number of flanges 812 on flange grip 810, it should be understood that any number of flanges 812 are possible. For instance, there may only be two, three, four, or any number of flanges 812 that can grasp catheter shaft 350. As an additional embodiment, flanges 812 may include a rubber material on a portion thereof to provide additional grip to catheter shaft 350. For example, the portion of flanges 812 that come into contact with catheter shaft 350 may include rubber material, that way when flanges 812 are securely tightened to catheter shaft 350, catheter shaft 350 is even more sturdily in place.

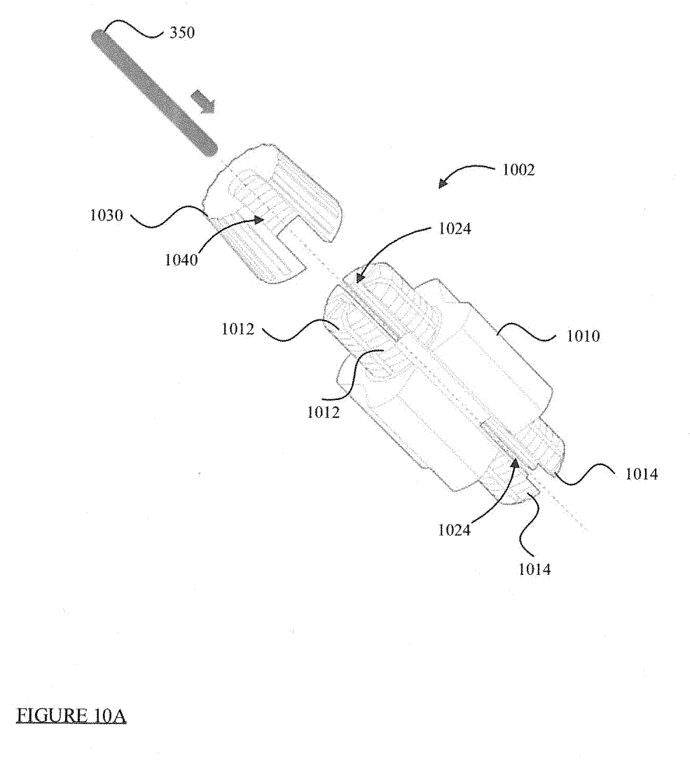

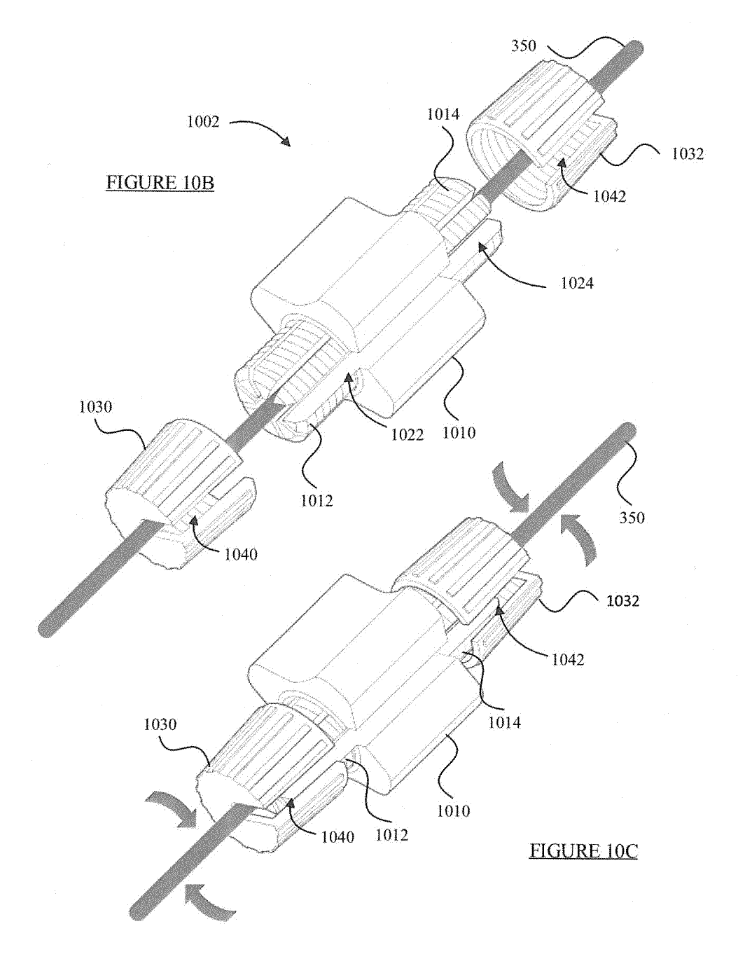

FIGS. 10A-C illustrate another embodiment of the flange catheter grip. In this example, double-sided flange catheter grip 1002 includes first cap 1030, second cap 1032, first flanges 1012, and second flanges 1014. First cap 1030 and second cap 1032 of which may operate similarly to cap 830 as described above, and first flanges 1012 and second flanges 1014 may act similarly to flanges 812 as described above. For example, first flanges 1012 and second flanges 1014 both include threads thereon that allow the threads on first cap 1030 and second cap 1032 to mate with.

Double-sided flange catheter grip 1002 include flanges 1012 and 1014 around bore 1024 to further secure catheter shaft 350 in place. For example, as discussed above with respect to the single-sided catheter grip 802, first flanges 1012 and second flanges 1014 may also have a resting position and a tightening position. For example, in the resting position catheter shaft 350 may be inserted or removed from first flanges 1012 and second flanges 1014, and in the tightening position first flanges 1012 and second flanges 1014 may secure catheter shaft 350 in place.

Additionally, although mating first cap 1030 with first flanges 1012 may properly secure catheter shaft 350 in place, a user may optionally mate second cap 1032 with second flanges 1014 for additional support. For example, double-sided flange catheter grip 1002 can be used for larger diameter catheter shafts in order to increase control for the user. Furthermore, double-sided flange catheter grip 1002 also includes first cap slit 1040 and second cap slit 1042, which may be formed similarly as discussed above with respect to slit 850. First flanges 1012 and second flanges 1014 are positioned at opposite ends of double-sided flange grip 1010, and may further include first flanges slit 1022 and second flanges slit 1024, which may be formed similarly to corresponding slit 852 as discussed above with respect to FIGS. 8A and 8C-D. Additionally, bore 1024 extends from first flanges 1012 to second flanges 1014, that way catheter shaft 350 is completely insertable through the entire double-sided flange grip 1010.

As a further embodiment, the grip of any of the catheter grip devices described above may include a rubber material thereon, either completely or only portions thereof, that provide even better grip for the user. As one example, the grip may be comprised of plastic, metal, etc. and the rubber may be positioned on a top portion of the grip to provide the user with additional grip when using the catheter grip. As another example, the rubber may be on the protrusion portion of the ridges only, that is, the recesses formed by the ridges may not include the rubber material. As a further example, the rubber may be on the ridges portion, the flat portion, or any combination thereof on the cap.

As a further example, the cap of any of the catheter grip devices described above may include a rubber material thereon as well, to provide additional grip support for the user. For instance, the cap may include the rubber material on a portion of the cap or over an entire external housing of the cap. The rubber material may only form on the protrusions of the ridges on the cap, that is, not inside the recesses. Alternatively, the rubber material may be on the protrusions and within the recesses of the ridges of the cap. As a further example, the rubber may be on the ridges portion, the flat portion, or any combination thereof on the cap.

As another embodiment, the grip and cap of any of the catheter grip devices described above may be permanently affixed to each other, such that the cap cannot be removed from the receiving portion or flanges of the catheter grip, depending on which device is used. For instance, as described above, the various catheter grip devices include recesses or slits that allow the user to insert the catheter shaft at prior to the cap being securely tightened to the receiving portion or flanges. In that regard, the threads may include a stopping device or mechanism that allows prohibits the user from completely removing the cap. For example, the cap may unscrew up until an end of the threads is reached, in which case the cap is stopped. In this scenario, the user can still insert the catheter shaft into the receiving recess or the bore, depending on the catheter grip device used, while the cap is attached, and then tighten the cap to fully secure the catheter shaft in place. This also has the advantage that the user does not need to worry about misplacing multiple pieces, and may also aid in transportation by only having to worry about one piece.

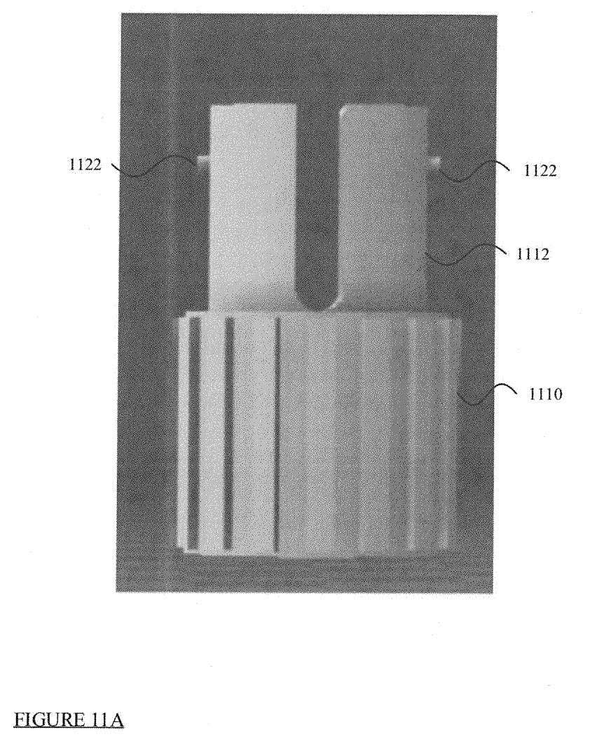

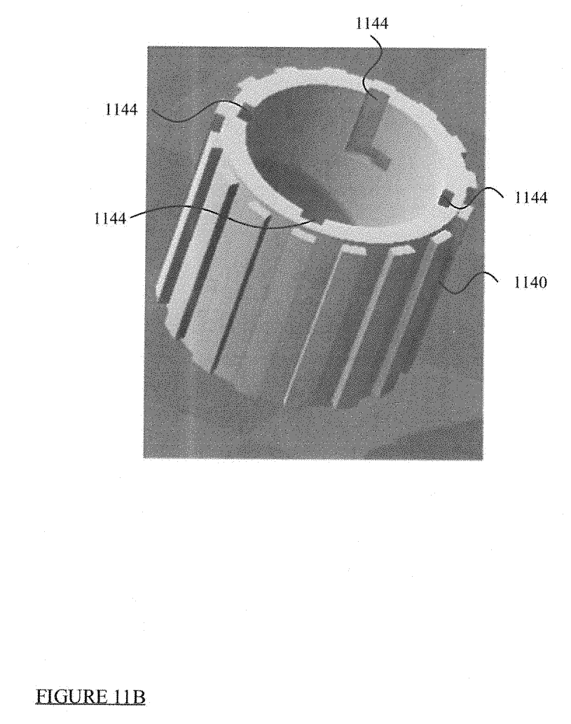

Other fastening mechanisms to fasten the cap to the receiving portion or flanges, depending on the device, are also possible. For example, the grip may have securing teeth on both sides that engage with a pawl in the cap to form a ratchet and tighten as the cap is depressed onto the grip. The grip may or may not have openings to accept the pawl section and also provide a release mechanism to allow the user to adjust the tension from the cap. As another embodiment and as shown in FIGS. 11A-B, grip 1110 has one or more protrusions or tabs 1122 that act as anchoring points for cap 1140. These tabs 1122 can be at various lengths, widths, and angles. Cap 1140 may have various and corresponding slots, notches, or openings 1122 at various lengths, widths, and angles to accept tabs 1122, thereby securing cap 1140 to grip 1110. Other attachment mechanisms between the grip and the cap are also possible, such as friction fit, press fit, etc.

As another embodiment of the present disclosure, different sized caps that have different interior depths may be manufactured so that the user can select the cap according to the size of the particular catheter shaft. The interior depth of the cap may be the distance between the base of the interior portion of the cap and the circumferential end of the cap. In this regard, catheter shafts with lesser width may require a user to select a cap with a deeper interior depth, this way the receiving portion of the grip is insertable into the cap enough so that the end of the cap is capable of coming into contact with the catheter shaft and thereby securing the catheter shaft in place. As a further example, a catheter shaft with greater width may require a user to select a cap with a more shallow interior depth, since the greater width catheter shaft covers greater space. In this example, the receiving portion may not extend too far into the interior of the cap because an end of the receiving portion will collide with the base of the interior portion of the cap. Additionally, the user can also select a full cap so that the tension is adjustable.

Advantages of the present disclosure include reducing the amount of strain on the user's hand muscles when operating and manipulating the catheter shaft. For instance, instead of the user having to grasp and manipulate the catheter shaft between his or her fingers, such as the thumb and index finger, the user is able to fully grasp the catheter grip and any manipulation of the catheter grip translates directly to the catheter shaft itself. In this regard, instead of all of the strain being on muscles within the hand, by being able to grasp the catheter grip the muscles in the wrist, forearm, and elbow may be utilized, thereby absorbing the strain on the muscles within the hand of the user. Furthermore, the present disclosure may also provide additional accuracy and overall control when manipulating the catheter shaft, thereby also increasing safety to the patient.

Most of the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A catheter grip, comprising:
a cap having an interior cavity;
a grip; and
a receiving portion extending from the grip, the receiving portion having an insertion area configured to receive a catheter shaft, wherein the interior cavity of the cap is fastened to the receiving portion of the grip.

2. The catheter grip of claim 2, wherein the grip has a first side and a second side, the receiving portion extending from the first side and a gripping portion being on the second side of the grip.

3. The catheter grip of claim 1, wherein the insertion area of the receiving portion is a recess, the recess extending perpendicular to the receiving portion.

4. The catheter grip of claim 3, wherein at least a portion of the recess includes a rubber insert to further secure the catheter grip in place.

5. The catheter grip of claim 3, wherein the recess extends from a first end of the receiving portion to a second end of the receiving portion.

6. The catheter grip of claim 3, wherein, when a catheter shaft is received within the recess of the receiving portion such that the catheter shaft rests on a recess base of the recess, a circumferential end of the cap contacts the catheter shaft such that the catheter shaft is positioned between the end of the cap and the recess base, thereby securing the catheter shaft in place.

7. The catheter grip of claim 1, wherein the insertion area is a bore that extends longitudinally from a first end of the receiving portion to a second end of the receiving portion.

8. The catheter grip of claim 7, wherein the bore further extends through the grip.

9. The catheter grip of claim 7, wherein the cap includes a cap bore that corresponds to the bore of the receiving portion when the receiving portion and the cap are at least partially fastened to each other.

10. The catheter grip of claim 9, wherein the cap includes a cut-out that defines an opening on at least a portion of the cap threads of the cap, the opening corresponding to at least a width of a catheter shaft, and the cut-out extending to the cap bore of the cap.

12. The catheter grip of claim 8, wherein the receiving portion of the grip includes flanges surrounding the bore, the flanges being configured to adjust a size of the bore, wherein the flanges have a resting position and a tightening position, wherein in the resting position the size of the bore has a first diameter adapted to receive the catheter shaft, and in the tightening position the size of the bore has a second diameter adapted to come into contact with the catheter shaft and thereby secure the catheter shaft in place.

13. The catheter grip of claim 1, wherein the cap is fastened to the receiving portion via at least one of threads, tab and notch, and teeth that secure to a pawl.

14. The catheter grip of claim 1, wherein a top of the receiving portion is tapered.

15. An apparatus, comprising:
a grip;
a first flanges extending from a first side of the grip; and
a second flanges extending from a second side of the grip, the second side being opposite the first side; and
a bore extending from the first flanges to the second flanges, wherein the bore is configured to receive a catheter shaft, and the first and second flanges are adapted to shift between a resting position and a tightening position.

16. The catheter grip of claim 15, further comprising:
a first cap having a first cap bore; and
a second cap having a second cap bore,
wherein when the first cap and the second cap are mated with the first flanges and the second flanges, respectively, and the position of the first cap bore and the second cap bore correspond to the bore of the first flanges and the second flanges.

17. A kit, comprising:
a first cap, the first cap having a first interior cavity;
a grip; and
a receiving portion extending from the grip, the receiving portion having an insertion area configured to receive a first catheter shaft, wherein the interior cavity of the cap is fastened to the receiving portion of the grip.

18. The kit of claim 17, further comprising a second cap, wherein the second cap has a second interior cavity, the second interior cavity being a different size from the first interior cavity.

19. The kit of claim 17, further comprising the first catheter shaft.

20. The kit of claim 17, wherein the first cap has a cap bore, and when the cap is fastened to the receiving portion, a position of the cap bore corresponds to an insertion area of the receiving portion.
